(19) United States
(12) Patent Application Publication
(10) Pub. No.: US 2003/0180270 A1
(43) Pub. Date: Sep. 25, 2003

(54) Methods and compositions for treating bone defects

(76) Inventor: Bruce Simon, Mountain Lakes, NJ (US)

Correspondence Address: HARNESS, DICKEY & PIERCE, P.L.C., P.O. BOX 828, BLOOMFIELD HILLS, MI, 48303, US

(21) Appl. No.: 10/372,999

(22) Filed: Feb. 24, 2003

Related U.S. Application Data
(60) Provisional application No. 60/358,772, filed on Feb. 22, 2002.

(51) Int. Cl. A61K 48/00

(52) U.S. Cl. 424/93.21

(57) ABSTRACT

Compositions and methods of treating bone and cartilage disorders and defects consisting of applying naturally secreted growth factors to a defect site. Specifically, the present invention provides a method of treating a bone or cartilage defect comprising:

culturing a living tissue, preferably skin tissue, so that the tissue secretes a plurality of growth factors; and

administering the growth factor to the site of the defect.

Compositions comprise a plurality of human growth factors secreted in a culture by living skin tissue. Preferably, the compositions additionally comprise a pharmaceutically-acceptable carrier. The growth factors are released onto a matrix or medium and subsequently processed for introduction to a defect site. In a preferred embodiment, the medium into which the factors are secreted is administered to the site of the defect.

### FIELD OF THE INVENTION

The present invention relates to compositions and methods for the treatment of bone and cartilage defects. More particularly, the present invention relates to such compositions derived from living skin tissue.

### BACKGROUND OF THE INVENTION

The present invention relates to the development of growth factors as therapeutics for the prevention and treatment of pathological conditions and other disorders involving bone or cartilage tissue, for example, osteoporosis, Paget's disease, fracture repair, periodontal disease, spinal fusion, and healing of bone and cartilage defects.

Currently available therapeutic agents known to stimulate or maintain bone are fluoride, calcitonin, parathyroid hormone, estrogen, selective estrogen receptor modulators (SERMs), bisphosphonates, and vitamin D. Fluoride clearly increases trabecular bone mass, but questions remain about the quality of the new bone formed and the side effects observed in some patients. Bisphosphonates, such as etidronate, alendronate, and risedronate, inhibit bone resorption. Other approaches for stimulating bone growth or maintaining bone mass involve the use of agents (e.g, parathyroid hormone) that activate osteoblasts (bone forming cells), or that interrupt resorption (e.g., calcitonin). One proposed therapeutic regimen is coherence therapy, where bone metabolic units are activated by oral phosphate administration and then resorption is inhibited by either bisphosphonates or calcitonin.

A number of growth factors have also been identified that stimulate osteoblasts and subsequently bone growth. The growth factors include those of the TGF-β superfamily (including the bone morphogenic proteins, or “BMPs”), PDGF, IGF-I, IGF-II, FGF, EGF, and VEGF. Numerous methods and procedures currently exist in the art for synthesizing or otherwise obtaining bone growth factors. However, many such growth factors are modified from naturally occurring growth factors and, as a result, have diminished effectiveness and/or side effects. In addition, they are usually synthesized without binding proteins or other factors that contribute to their in vivo effectiveness.

### SUMMARY OF THE INVENTION

The present invention provides a method of treating bone and cartilage defects and diseases in human or other animal subjects consisting of applying naturally secreted growth factor proteins to a defect site. Specifically, the present invention provides a method of treating a bone or cartilage defect in a human or other animal subject comprising:

culturing a living tissue so that said tissue secretes a plurality of growth factors; and

administering said growth factors to said subject at the site of said defect.

Preferably, the tissue is cultured so that it secretes growth factors in essentially quantities and ratios similar to that seen in vivo. Preferably, the tissue is skin, cartilage or other mesodermal tissue. A preferred embodiment is for the treatment of bone or cartilage defects. The growth factors are released onto a matrix or medium and subsequently processed for introduction to a bone or cartilage defect site. In a preferred embodiment, the medium into which the factors are secreted is administered to the site of the bone defect.

The invention also provides compositions for generating new bone growth comprising a plurality of human growth factors secreted in a culture by living skin tissue. Preferably, the composition comprises growth factors in ratios essentially present in the skin culture, preferably essentially as present in vivo. Preferably, the compositions comprise a pharmaceutically-acceptable carrier, such as hyaluronic acid, gelatin, collagen, cellulose ether, and osteoconductive materials.

It has been found that the compositions and methods of this invention afford benefits over compositions and methods among those known in the art. Such benefits include enhanced effectiveness, reduced side effects, ease of administration, and reduced cost of therapy. Specific benefits and embodiments of the present invention are apparent from the detailed description set forth herein. It should be understood that the detailed description and specific examples, while indicating embodiments of the invention, are intended for purposes of illustration only and are not intended to limit the scope of the invention.

### DETAILED DESCRIPTION

The present invention involves the treatment of bone or cartilage defects in humans or other animal subjects. Specific materials to be used in the invention must, accordingly, be pharmaceutically acceptable. As used herein, such a “pharmaceutically acceptable” component is one that is suitable for use with humans and/or animals without undue adverse side effects (such as toxicity, irritation, and allergic response) commensurate with a reasonable benefit/risk ratio.

Bone and Cartilage Defects:

The compositions and methods of this invention may be used to repair bone or cartilage defects. In one embodiment, the compositions and methods are for the treatment of cartilage defects. In another embodiment, the compositions and methods are for the treatment of bone defects. As referred to herein such “bone defects” include any condition involving skeletal tissue which is inadequate for physiological or cosmetic purposes. Such defects include those that are congenital, the result of disease or trauma, and consequent to surgical or other medical procedures. Specific defects include those resulting from bone fractures, osteoporosis, spinal fixation procedures, and hip and other joint replacement procedures. (As used herein, the word “include,” and its variants, is intended to be non-limiting, such that recitation of items in a list is not to the exclusion of other like items that may also be useful in the materials, compositions, devices, and methods of this invention. Also, as used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.)

Culturing of Tissue:

The methods of the present invention comprise culturing a living tissue. Such tissues include skin and cartilage tissues. A preferred embodiment comprises the culturing of skin tissues. Such skin tissue may be from human or other animal sources, preferably from human. The skin tissue used in the current invention may be obtained by appropriate biopsy or upon autopsy. The skin tissue is grown on a substrate and the skin cells are inoculated onto a scaffold in vitro. The skin cells are preferably stromal cells, derived from bone marrow or, preferably, from loose connective tissue. Stromal cells useful herein include endothelial cells, pericytes, macrophages, monocytes, leukocytes, plasma cells, and mast cells, as well as progenitor cells of keratinocytes, chondrocytes and adipocytes. The stromal cells may also comprise fibroblasts with or without additional cells and/or other elements.

Culturing of the skin tissue is effected by any means by which the viability of the tissue is maintained and growth factors are produced. Preferably, in such methods, the skin tissue is cultured in a growth medium (herein “medium”), and produces an extracellular matrix (herein “matrix”) comprising collagen and other proteins. Preferably, the matrix is secreted by the tissue into the medium or onto a scaffold with the various growth factor proteins found both in the matrix and the medium used to treat bone or cartilage defects. Culturing methods among those useful herein are disclosed in U.S. Pat. No. 6,039,760, Eisenberg, issued Mar. 21, 2000; and U.S. Pat. No. 6,284,284, Naughton, issued Sep. 4, 2001; both of which are incorporated by reference herein.

In a preferred embodiment, the tissue is cultured on a three dimensional platform. In such an embodiment, the matrix secreted by skin cells comprises type I and type III collagens, decorin, growth factors of the TGF-βsuperfamily, PDGF, IGF-I, IFG-II, FGF, EGF, VEGF, and various other extracellular matrix proteins in quantities and ratios similar to that existing in vivo. As referred to herein, the “TGF-β superfamily” refers to TGF-β and related cytokines, including the TGF-β subfamily (e.g., TGF-β1 and TGF-β2); the DVR (decapentaplegic and vegetal-1-related) subfamily (e.g., the BMPs); the activin/inhibin subfamily; and the GDNF subfamily.

The three-dimensional support used to culture tissue may comprise any material and/or shape that allows cells of the tissue to attach to it, preferably also allowing the cells to grow in more than one layer. Such materials are preferably selected from the group consisting of polyamides, polyesters, polystyrene, polypropylene, polyacrylates, polyvinyl compounds (e.g., polyvinylchloride), polycarbonate, polytetrafluorethylene, nitrocellulose, cotton, polyglycolic acid, cat gut sutures, cellulose, gelatin, dextran, collagen, chitosan, hyaluronic acid, and mixtures thereof. In one embodiment, these materials are woven into a mesh to form a three-dimensional framework. In another embodiment, the materials are used to form other types of three-dimensional frameworks, such as collagen sponges. Optionally, the framework is pre-treated prior to inoculation of stromal cells in order to enhance the attachment of stromal cells. For example, prior to inoculation with stromal cells, nylon frameworks can be treated with 0.1 M acetic acid, and incubated in polylysine, serum or serum components, or collagen to coat the nylon. Polystyrene can be similarly treated using sulfuric acid. A preferred nylon mesh which can be used in accordance with the invention is Nitex®, a nylon filtration mesh having an average pore size of 210 microns and an average nylon fiber diameter of 90 microns (sold by Sephar America, Depew, N.Y., U.S.A.).

In one embodiment, stromal cells comprising fibroblasts derived from adult or fetal tissue, with or without other cells and elements described below, are inoculated onto the framework. These fibroblasts may be derived from organs such as skin, liver, and pancreas, which can be obtained by biopsy, where appropriate, or upon autopsy. In a preferred embodiment, fetal neonatal fibroblasts can be obtained in high quantity from foreskin. Fibroblasts may be readily isolated by disaggregating an appropriate organ or tissue which is to serve as the source of the fibroblasts. This can be readily accomplished using techniques including those known in the art. For example, the tissue or organ can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mincing the tissue and treating the minced tissue with any of a number of digestive enzymes either alone or in combination. Such enzymes include trypsin, chymotrypsin, collagenase, elastase, hyaluronidase, pronase, dispase, and mixtures thereof. Mechanical disruption can also be accomplished by a number of methods including the use of grinders, blenders, sieves, homogenizers, pressure cells, and sonicators.

Once the tissue has been reduced to a suspension of individual cells, the suspension can be fractionated into subpopulations from which the fibroblasts and/or other stromal cells and/or elements can be obtained. This also may be accomplished using standard techniques for cell separation including cloning and selection of specific cell types; selective destruction of unwanted cells (negative selection); separation based upon differential cell agglutinability in the mixed population; freeze-thaw procedures; differential adherence properties of the cells in the mixed population; filtration; conventional, differential, and zonal centrifugation; centrifugal elutriation (counter-streaming centrifugation); unit gravity separation; countercurrent distribution; electrophoresis; and fluorescence-activated cell sorting.

The isolation of fibroblasts, for example, can be carried out as follows. Fresh tissue samples are thoroughly washed and minced in Hanks' balanced salt solution (HBSS) in order to remove serum. The minced tissue is incubated from 1-12 hours in a freshly prepared solution of a dissociating enzyme such as trypsin. After such incubation, the dissociated cells are suspended, pelleted by centrifugation and plated onto culture dishes. All fibroblasts will attach before other cells, therefore, appropriate stromal cells can be selectively isolated and grown. The isolated fibroblasts can then be grown to confluency, lifted from the confluent culture and inoculated onto the three-dimensional framework.

Inoculation of the three-dimensional framework with a high concentration of stromal cells, e.g., approximately 10⁶ to 5×10⁷ cells/ml, will result in the establishment of the three-dimensional stromal support in shorter periods of time. In addition to fibroblasts, other cells can be added to form the three-dimensional stromal cell culture-producing extracellular matrix. For example, other cells found in loose connective tissue may be inoculated onto the three-dimensional support framework along with fibroblasts. Such cells include endothelial cells, pericytes, macrophages, monocytes, plasma cells, and mast cells, and progenitor cells of adipocytes, keratinocytes, and chondrocytes. These stromal cells can be readily derived from appropriate organs such as skin, liver, etc., using methods known, such as those discussed above.

In one embodiment of the present invention, stromal cells which are specialized for the particular tissue to be cultured can be added to the fibroblast stroma for the production of a tissue type specific extracellular matrix. For example, dermal fibroblasts can be used to form the three-dimensional subconfluent stroma for the production of skin-specific extracellular matrix in vitro. Alternatively, stromal cells of hematopoietic tissue including fibroblast endothelial cells, macrophages/monocytes, adipocytes and reticular cells, can be used to form the three-dimensional subconfluent stroma for the production of a bone marrow-specific extracellular matrix in vitro. Hematopoietic stromal cells can be readily obtained from the “buffy coat” formed in bone marrow suspensions by centrifugation at low forces, e.g., 3000G. Stromal cells of liver include fibroblasts, Kupffer cells, and vascular and bile duct endothelial cells. Similarly, glial cells can be used as the stroma to support the proliferation of neurological cells and tissues. Glial cells for this purpose can be obtained by trypsinization or collagenase digestion of embryonic or adult brain.

Once the skin cells have been isolated and inoculated onto the scaffold, the skin cells grow on the scaffold and secrete an extracellular matrix comprised of growth factors, glycosaminoglycans, and other extracellular matrix proteins onto the scaffold and into the surrounding medium. Cell growth is aided by incubation of the scaffold in an appropriate nutrient medium under physiologic conditions favorable for cell growth. Appropriate media that may be used include RPMI 1640, Fisher's, Iscove's, and McCoy's solution.

Growth Factors:

The matrix and medium preferably comprise growth factors secreted by the tissue. Such growth factors include those selected from the group consisting of the TGF-β superfamily, PDGF, IGF-I, IGF-II, FGF, EGF, VEGF, and mixtures thereof. Methods of this invention comprise administering the growth factors present in the matrix, the medium, or both, to the bone or cartilage defect site to effect repair or growth. The growth factors may be extracted from the medium or matrix, or the medium or matrix may be administered directly to the site of the defect. In one embodiment, this invention provides a composition in powder form comprising a bone growth factor extracted from the tissue culture. Preferably, the composition comprises two or more, preferably three or more, growth factors. In a preferred embodiment, the bone growth factor is extracted from the medium, matrix, or both the medium and the matrix. As referred to herein, a “powder” is any form of the composition which is substantially dry, i.e., containing little or no water. Such powders may be formed from the matrix or medium by a variety of methods, including those known in the art. A preferred method is lyophilization. Such powder compositions may be administered directly to the site of the defect, or mixed with saline or another suitable carrier prior to administration

Compositions:

The present invention comprises compositions comprising a plurality of growth factors secreted by the tissue culture. Preferably, a plurality of factors comprises at least 10, more preferably at least about 20, factors secreted by the skin culture. Preferably, the composition comprises factors of types and in quantitative ratios substantially similar to those found in the skin culture. The present invention also provides methods of making a composition for the treatment of a bone or cartilage defect, comprising:

(a) culturing a living tissue so that said tissue secretes a plurality of human growth factors;

(b) isolating said human growth factors; and

(c) forming a mixture of said growth factors and a pharmaceutically acceptable carrier. Preferably, the culturing step comprises growing skin tissue to form a matrix in contact with a medium, and the isolating step comprises separation of said matrix from said medium.

Preferably, the compositions comprise a pharmaceutically-acceptable carrier. In one embodiment, the carrier comprises the matrix. In another embodiment, the carrier comprises the medium. Accordingly, one embodiment of this invention comprises the administration of the matrix to the site of a bone or cartilage defect, where the matrix comprises a plurality of growth factors. Another embodiment of this invention comprises the administration of the medium to the site of a defect, where the medium comprises a plurality of growth factors. Optionally, the matrix or medium comprises additional carrier materials. Also optionally, the matrix or medium comprises additional bone growth active materials.

In a preferred embodiment, the composition is lyophilized or otherwise processed to form a dry powder which may be administered directly to the site of the bone or cartilage defect, or mixed with saline or another suitable carrier prior to administration. In a preferred embodiment, the concentration of the bone growth factors after mixture with saline or other carrier is greater than the concentration of the growth factors in the medium.

Preferred pharmaceutically acceptable carriers include saline, hyaluronic acid, cellulose ethers (such as carboxymethyl cellulose), collagen, gelatin, an osteoconductive carrier, and mixtures thereof. Osteoconductive carriers include allograft bone particles, demineralized bone matrix, calcium phosphate, calcium sulfate, hydroxyapatite, polylactic acid, polyglycolic acid and mixtures thereof. The compositions may optionally comprise other bone growth active materials, such as other growth factors, hormones (e.g., estrogen, calcitonin, parathyroid hormone, selective estrogen receptor modulators), and phosphonates (e.g., bisphosphonates).

The compositions of the present invention may be made in any of a variety of ways. In one embodiment, the matrix or medium is reduced to a powder, and the powder is coated on, or otherwise mixed with, a carrier. In another embodiment, the matrix or medium is mixed with the carrier, and the mixture is lyophilized.

In a preferred embodiment, the carrier comprises an osteoconductive carrier selected from the group consisting of calcium phosphate, hydroxyapatite, calcium sulfate, and mixtures thereof, preferably as a hardening paste. In one embodiment, the growth factors are mixed with the carrier during formation of the hardening paste, and the paste applied to the site of the bone or cartilage defect. In another embodiment, the growth factors are mixed with the carrier during formation of the hardening paste, the paste allowed to harden, and then the paste is broken up into small particles before administration to the site of the defect.

The following non-limiting examples illustrate the compositions and methods of the present invention.

### EXAMPLE 1

Fibroblast cells are established on a sterilized nylon mesh and placed in a suitable medium, such as RPNI 1640, Fisher's, Iscove's, or McCoy's solution. The fibroblast cells begin to grow into the meshwork openings within 6-9 days of incubation. As the fibroblast cells grow they deposit extracellular matrix onto the mesh and into the surrounding medium. The extracellular matrix comprises, among other extracellular components, growth proteins. Specifically, the growth proteins present in the matrix are those of the TGF-β superfamily, PDGF, IGF-I, IFG-II, FGF, EGF, and VEGF.

The matrix comprising the above growth factors is lyophilized, producing a powder comprising the growth factors and other extracellular products. The powder is combined with saline and introduced through a syringe to a tibia fracture site. The fracture site heals in eight weeks as opposed to sixteen weeks.

In the above Example, hyaluronic acid, collagen, gelatin, or methyl cellulose are combined with saline prior to injection, with substantially similar results.

### EXAMPLE 2

Skin fibroblasts are isolated by mincing dermal tissue, trypsinizing for 2 hours, and separating the cells into a suspension by physical means. The fibroblasts are grown to confluency in 25 cm² Falcon tissue culture dishes and are fed RPMI 1640 (Sigma, MO) supplemented with 10% bovine serum, fungizone, gentamicin, and penicillin/streptomycin. The fibroblasts are lifted by mild trypsinization and the cells are plated onto a nylon filtration mesh, the fibers being approximately 90 μm in diameter, and are assembled into square weave with a mesh opening of 210 μm. The mesh is pretreated with a mild acid wash and incubated in polylysine and FBS. Adherence of the fibroblasts occurs in 3 hours and the fibroblasts begin to stretch across the mesh openings within 5 to 7 days of initial inoculation. The fibroblasts are metabolically active, secrete an extracellular matrix, and rapidly form a dermal equivalent consisting of active fibroblasts and collagen.

The medium comprises, among other substances, growth factors including those of the TGF-β superfamily, PDGF, IGF-I, IFG-II, FGF, EGF, and VEGF. The medium is then coated onto an spinal implant allograft, and lyophilized. The allograft is then used in spinal fusion surgery, resulting in enhanced fusion of the spine.

### EXAMPLE 3

Samples of oral mucosal tissue are obtained from orthodontic surgical species. The tissue is washed three times with fresh MEM containing antibodies (2 ml of antibiotic antimycotic solution and 0.01 ml of gentamicin solution), cut into small pieces and then washed with 0.02% EDTA. 0.25% trypsin (in PBS without Ca⁺⁺¹ or Mg⁺⁺) and refrigerated at 4° C. overnight. The tissues are then removed and placed in fresh trypsin solution, and gently agitated until the cell appears to form a single-cell suspension. The single-cell suspension is then diluted in MEM containing 10% heat inactivated fetal bovine serum and centrifuged at 1400×g for 7 minutes. The supernatant is decanted and the pellet containing mucosal epithelial cells is placed into a seeding medium. The medium consists of DMEM with 2% Ultrosen G, 1× L-glutamine, 1× non-essential amino acids, penicillin and streptomycin.

The cells are then seeded onto a three dimensional mesh framework. The mesh is soaked in 0.1M acetic acid for 30 minutes and treated with 10 mM polylysine suspension for 1 hour. The mesh is placed in a Corning 25 cm tissue culture flask, floated with an additional 5 ml of medium, and allowed to reach subconfluence, being fed at 3 day intervals. Cultures are maintained in DMEM complete medium at 37° C. and 5% CO₂ in a humidified atmosphere and are fed with fresh medium every 3 days.

As the cells proliferate on the mesh they excrete extracellular matrix onto the mesh and into the surrounding medium. The medium comprising the above growth factors is lyophilized to form a powder comprising factors of the TGF-β superfamily, PDGF, IGF-I, IFG-II, FGF, EGF, VEGF and other growth factors. The powder is then mixed with a hardening paste comprising calcium phosphate and hydroxyapatite, and the paste applied (before hardening) at the site of a hip implant. Bone growth is observed within three weeks.

What is claimed is:

1. A method of treating a bone or cartilage defect in a human or other animal subject comprising:
culturing a living tissue so that said tissue secretes a plurality of growth factors; and
administering said growth factors to said subject at the site of said defect.

2. The method according to claim 1, wherein said culturing step comprises growing skin tissue to form a matrix in contact with a medium.

3. The method according to claim 2, wherein said growth factors are secreted into said matrix, and said administering step comprises administration of said matrix to said site.

4. The method according to claim 2, wherein said growth factors are secreted into said medium, and said administering step comprises administration of said medium to said site.

5. The method according to claim 3, wherein said matrix comprising said growth factors is lyophilized prior to said administering step.

6. The method according to claim 4, wherein said medium comprising said growth factors is lyophilized prior to said administering step.

7. The method according to claim 1, wherein said tissue comprises fibroblast cells.

8. The method according to claim 1, wherein said tissue comprises stromal cells derived from loose connective tissue or bone marrow.

9. The method according to claim 8, wherein said stromal cells are endothelial cells, pericytes, macrophages, monocytes, leukocytes, plasma cells, and mast cells, and progenitors of keratinocytes, chondrocytes and adipocytes.

10. The method according to claim 2, wherein said growing step is performed by growing stromal cells of said tissue on a scaffold.

11. The method according claim 1, wherein said human growth factors comprise growth factors selected from the group consisting of the TGF-β superfamily, PDGF, IGF-I, IFG-II, FGF, EGF, VEGF, and mixtures thereof.

12. The method according to claim 4, further comprising the steps of lyophilizing said medium and mixing said lyophilized medium with a suitable carrier prior to said administering step.

13. The method according to claim 12, wherein said carrier comprises saline.

14. The method according to claim 12 wherein said carrier comprises hyaluronic acid.

15. The method according to claim 12, wherein said carrier comprises cellulose ether.

16. The method according to claim 12, wherein said carrier comprises collagen.

17. The method according to claim 12, wherein said carrier comprises an osteoconductive carrier.

18. The method according to claim 17, wherein said osteoconductive carrier comprises a carrier selected from the group consisting of allograft bone particles, demineralized bone matrix, calcium phosphate, hydroxyapatite, calcium sulfate, polylactic acid, polyglycolic acid, and mixtures thereof.

19. The method according to claim 4, further comprising the steps of forming a composition comprising an osteoconductive carrier coated with said medium, and lyophilizing said composition before said administering step.

20. The method according to claim 19, wherein said osteoconductive carrier comprises a carrier selected from the group consisting of allograft bone particles, demineralized bone matrix, calcium phosphate, hydroxyapatite, calcium sulfate, polylactic acid, polyglycolic acid, and mixtures thereof.

21. The method according to claim 4, further comprising the steps of forming a mixture of said medium with a carrier, and lyophilizing said mixture before said administering step.

22. A composition for generating new bone growth comprising a plurality of human growth factors secreted in a culture by living tissue.

23. A composition according to claim 24, additionally comprising a pharmaceutically-acceptable carrier.

24. A composition according to claim 23, comprising said growth factors in a medium isolated from said culture.

25. A composition according to claim 23, wherein said carrier comprises a material selected from the group consisting of hyaluronic acid, gelatin, collagen, cellulose ether, osteoconductive carriers, and mixtures thereof.

26. A composition according to claim 24, wherein said carrier comprises an osteoconductive carrier selected from the group consisting of allograft bone particles, demineralized bone matrix, calcium phosphate, hydroxyapatite, calcium sulfate, polylactic acid, polyglycolic acid, and mixtures thereof.

27. The composition according to claim 22, wherein said composition is lyophilized.

28. A method of treating a bone or cartilage defect in a human or other animal subject comprising:
(a) forming a mixture comprising a composition according to claim 22 and a pharmaceutically acceptable carrier; and
(b) introducing said mixture to the site of said defect.

29. A method of making a composition for the treatment of a bone or cartilage defect, comprising:
(a) culturing a living tissue so that said tissue secretes a plurality of human growth factors;
(b) isolating said human growth factors; and
(c) forming a mixture of said growth factors and a pharmaceutically acceptable carrier.

30. A method according to claim 29, wherein said culturing step comprises growing skin tissue to form a matrix in contact with a medium, and said isolating step comprises separation of said matrix from said medium.

31. A method according to claim 29, wherein said mixture comprises said growth factors of types and quantitative ratio essentially identical to the types and ratio of said factors secreted in said culturing step.

32. A method according to claim 29, additionally comprising the step of lyophilizing said mixture.
